(19) United States
(12) Patent Application Publication
(10) Pub. No.: US 2017/0079629 A1
(43) Pub. Date: Mar. 23, 2017

(54) DIVERTICULUM INVERSION USING A CLIP PLACEMENT DEVICE

(71) Applicant: Empire Technology Development LLC, Wilmington, DE (US)

(72) Inventors: Sam Malanowski, San Francisco, CA (US); Todd Dickson, Santa Clara, CA (US)

(21) Appl. No.: 14/859,108

(22) Filed: Sep. 18, 2015

(51) Int. Cl. A61B 17/00; A61B 17/128; A61B 17/122; A61B 17/10; A61B 17/08

(52) U.S. Cl. CPC A61B 17/0057; A61B 17/10; A61B 17/083; A61B 17/122; A61B 17/1285; A61B 2017/00862; A61B 2017/2215

(57) ABSTRACT

Several embodiments disclosed herein relate to apparatus and methods for treating a diverticulum. In some examples, disclosed is a clip placement device for diverticulum inversion. The clip placement device can include a clip tube, a clip, a clip attachment structure, and an expansion apparatus. A method for clip placement for diverticulum inversion can also be included. The method can include positioning the distal end of a clip placement device along an outer wall of a colon at a diverticulum, inverting the diverticulum, and advancing a portion of the clip placement device into the inverted diverticulum. The diverticulum can then be expanded with the expansion apparatus. The method can then include engaging the clip with the diverticulum, and withdrawing the clip placement device in a proximal direction such that that clip attachment structure disengages from the clip from the clip placement device.

### BACKGROUND

An outpouching of the colon or other body lumen, called a diverticulum, can become the site for inflammation known as diverticulitis, microperforation and/or bleeding. Current treatments may involve the surgical removal of segments of the body lumen. For extreme cases of diverticulitis, treatment can involve colon resection and placement of a colostomy. This approach results in significant healthcare costs and substantial pain for patients.

### SUMMARY

Disclosed is a method of clip placement for diverticulum inversion. In some embodiments the method includes positioning the distal end of a clip placement device along an outer wall of a colon at a diverticulum. In some embodiments, the method includes inverting the diverticulum into the lumen of the colon with a distal end of a pusher structure within the clip placement device. In some embodiments, the methods includes advancing a portion of the clip placement device into the inverted diverticulum, wherein the clip placement device includes the pusher structure, a clip tube, a clip, a clip attachment structure configured to reversibly engage the clip at the distal end of the clip tube, and an expansion apparatus. In some embodiments, the method includes expanding the diverticulum with the expansion apparatus such that the volume of the diverticulum increases. In some embodiments, the method includes engaging the clip with the diverticulum. In some embodiments, the method includes withdrawing the clip placement device in a proximal direction such that the clip attachment structure disengages the clip from the clip placement device.

In other embodiments, the method includes an expansion apparatus that can be configured to release a gas to expand the diverticulum. In other embodiments, the released gas is an inert and/or biocompatible gas such as CO₂, O₂, or N₂.

In other embodiments, the method includes an expansion apparatus is configured to release a fluid to expand the diverticulum. In other embodiments, the fluid is a biocompatible liquid such as water or saline. In other embodiments, the fluid is a solution that can include anti-inflammatory drugs, growth factors, or antibiotics.

In other embodiments, the method includes an expansion apparatus that includes a retractable structure configured to expand the diverticulum. In other methods, the expansion apparatus comprises an elastic material. In other methods, the elastic material is selected from an elastic polymer or metal such as nitinol, cobalt-chromium, polyurethane, polyethylene terephthalate, and polyethyleneoxide.

In other embodiments, the expansion apparatus can include a braided nitinol cage, a spherical structure, a football structure, a malecot, a spiral wire, or curved wires.

Disclosed is a clip placement device for diverticulum inversion. In some embodiments, the clip placement device includes a clip tube, a clip, a clip attachment structure, that is configured to reversibly engage the clip at the distal end of the clip tube, and an expansion apparatus.

In some embodiments, the clip placement device includes an expansion apparatus that is configured to release a gas to expand the diverticulum. In other embodiments, the gas is an inert and/or biocompatible gas such as CO₂, O₂, or N₂.

In some embodiments, the clip placement device includes an expansion apparatus that is configured to release a fluid to expand the diverticulum. In other embodiments the fluid is a biocompatible liquid such as water or saline. In other embodiments, the fluid is a solution that can include anti-inflammatory drugs, growth factors, or antibiotics.

In some embodiments, the clip placement device includes an expansion apparatus including a retractable structure configured to expand the diverticulum.

In some embodiments, the clip placement includes an expansion apparatus is composed of an elastic material. In other embodiments, the elastic material is selected from an elastic polymer or metal such as nitinol, cobalt-chromium, polyurethane, polyethylene terephthalate, and polyethyleneoxide.

In some embodiments, The clip placement device includes an expansion apparatus composed of a braided nitinol cage, a spherical structure, a football structure, a malecot, a spiral wire, or curved wires.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A illustrates a side view of an embodiment of a device for inverting diverticulum with all of the components of the device exposed.

FIG. 1B illustrates a side view of the device illustrated in FIG. 1A with a few of the components retracted.

FIGS. 1C-K illustrate a plurality of views of a method for treating diverticulum disease using the device of FIG. 1A.

FIG. 1L illustrates a flowchart of an embodiment of the method for treating diverticulum disease illustrated in FIGS. 1C-K.

FIG. 2A illustrates a side view of another embodiment of a device for inverting diverticulum.

FIGS. 2B-H illustrate a plurality of views of a method for treating diverticulum disease using the device of FIG. 2A.

FIG. 3A illustrates a side view of another embodiment of a device for inverting diverticulum.

FIG. 3B illustrates a top view of an embodiment of a closure clip in a relaxed configuration wherein the closure clip can be used with the devices illustrated in FIGS. 1A, 2A, and 3A.

FIG. 3C illustrates a side view of the closure clip of FIG. 3B when it is loaded on any of the devices illustrated in FIGS. 1A, 2A, and 3A.

FIG. 3D illustrates a side view of the closure clip of FIG. 3B as it is delivered to a target site.

FIG. 3E illustrates a side view of an embodiment of a clip tube of the device of FIG. 3A.

FIG. 3F illustrates the clip tube of FIG. 3E that is loaded with the closure clip illustrated in FIG. 3D.

FIGS. 3G-I illustrate a plurality of views of the distal end of an embodiment of a push rod of the device of FIG. 3A.

FIG. 3J illustrates a side view of the clip tube of FIG. 3E and push rod of FIGS. 3G-I that is loaded with the closure clip illustrated in FIG. 3D.

FIG. 3K illustrates a side view of the apparatus of 3J where the closure clip is configured for delivery as illustrated in FIG. 3E.

FIG. 3L illustrates an embodiment of a sheath of the device of FIG. 3A.

FIGS. 3M-S illustrate a plurality of views of a method for treating diverticulum using the device of FIG. 3A.

FIGS. 4A-B are side views of a device for treating diverticulum disease in a collapsed inverted diverticula.

FIG. 5 is a side view of the device for diverticulum expansion with fluid release distal end for treating diverticulum disease.

FIG. 6 is a side view of the device for diverticulum expansion with expandable distal end for treating diverticulum disease.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Disclosed herein are methods and devices for treating diverticular disease. In particular, disclosed are methods and devices for clip placement for diverticulum inversion. The method can include positioning the distal end of a clip placement device along an outer wall of a colon at a diverticulum, inverting the diverticulum into the lumen of the colon with a distal end of a pusher structure within the clip placement device, advance a portion of the clip placement device into the inverted diverticulum, expanding the diverticulum with the expansion apparatus, engaging the clip with the diverticulum, and then withdrawing the clip placement device in a proximal direction such that the clip attachment structure disengages the clip from the clip placement device. The device can include a clip tube, a clip, a clip attachment structure, and an expansion apparatus.

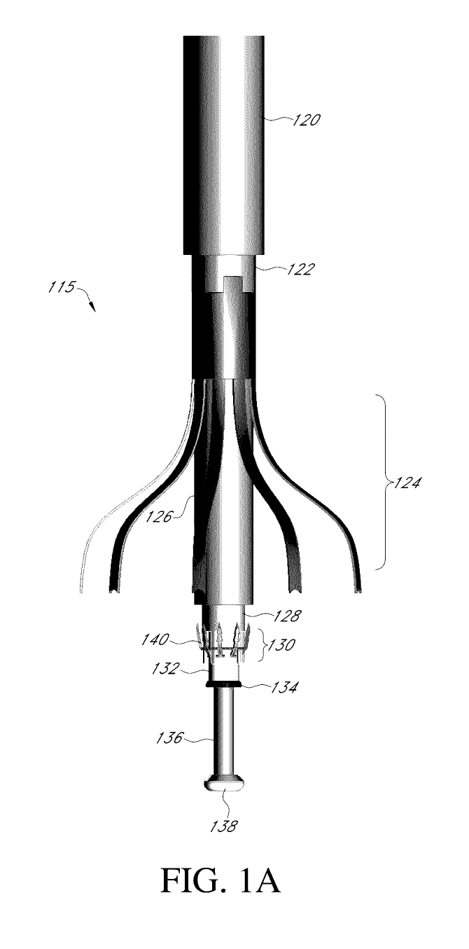

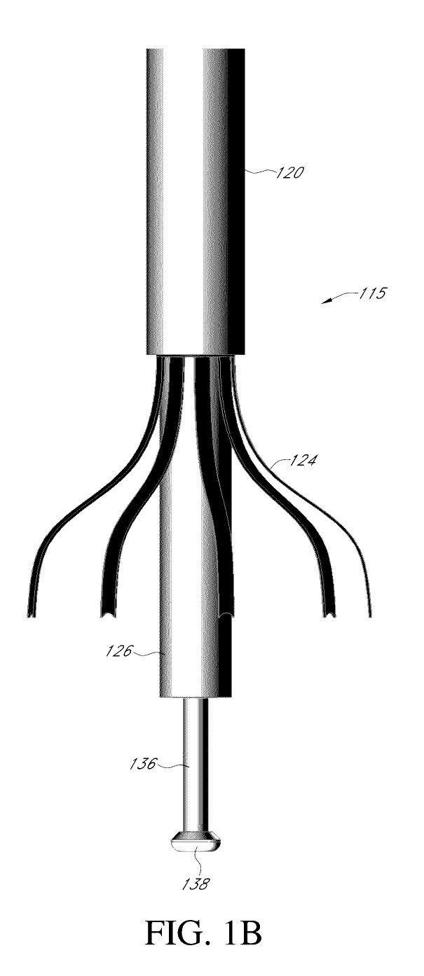

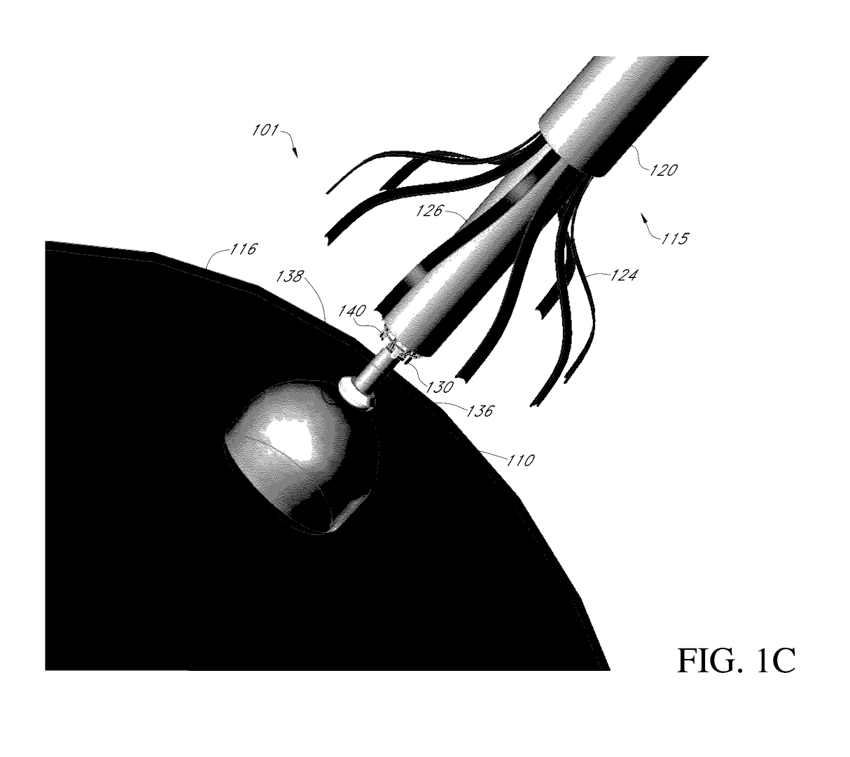

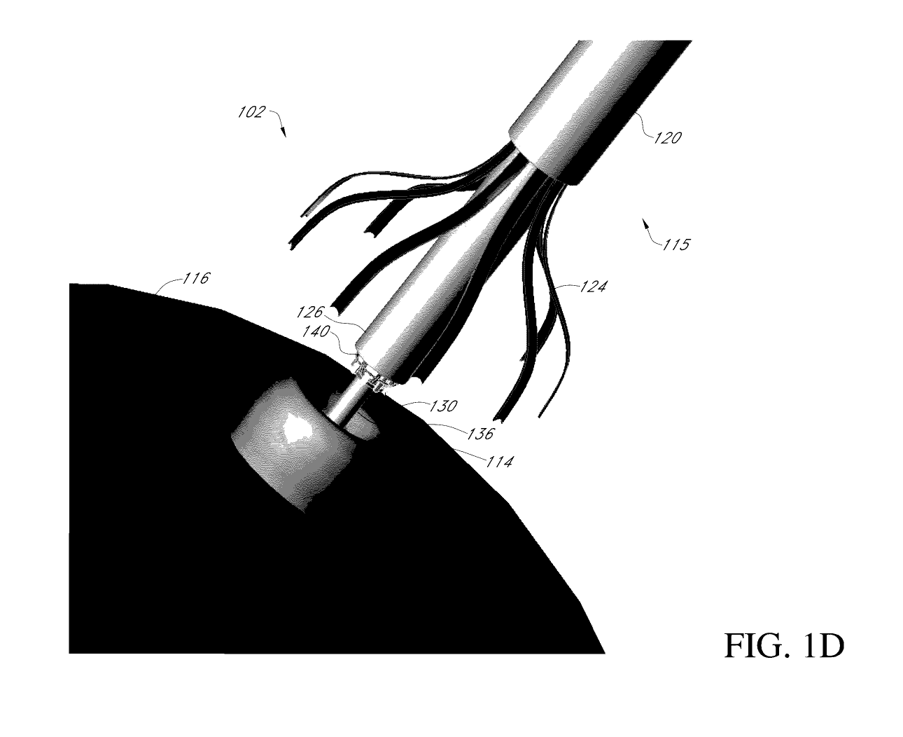

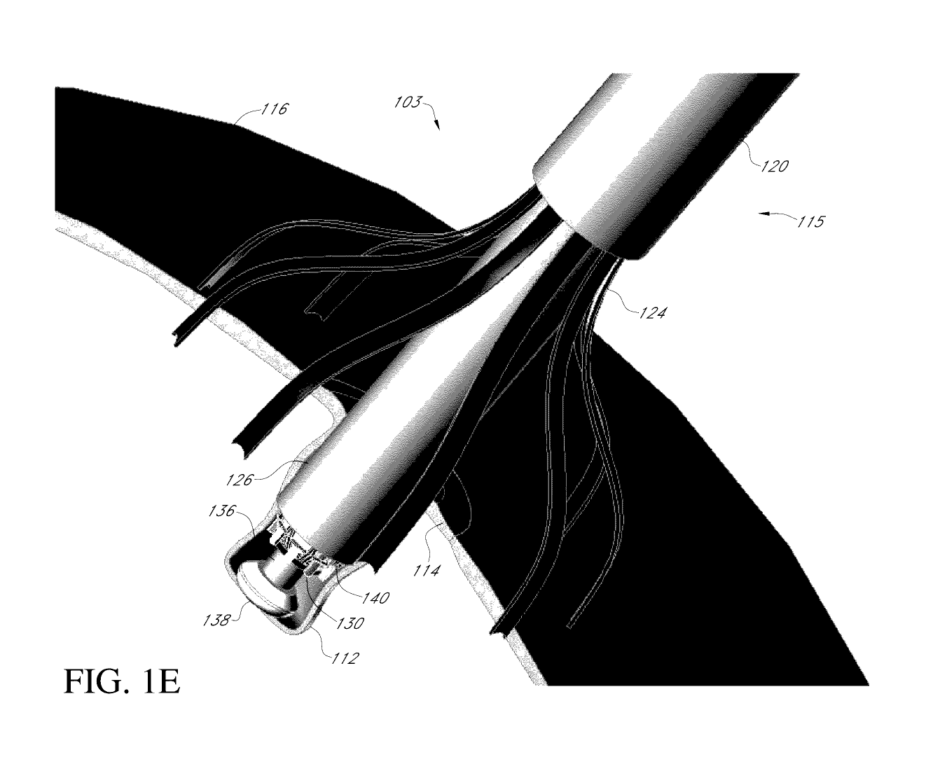

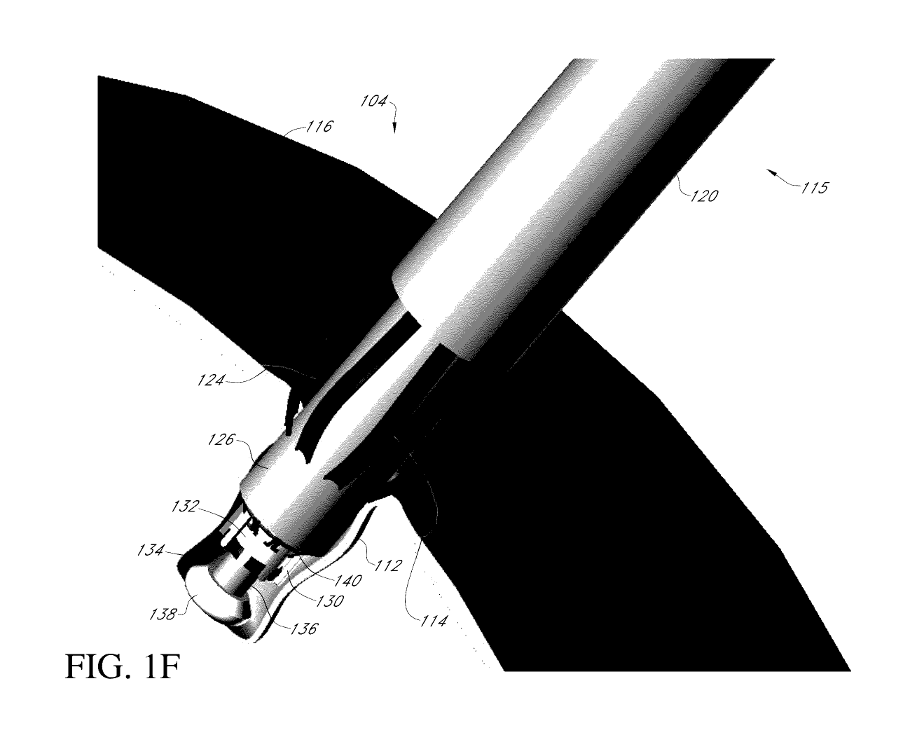

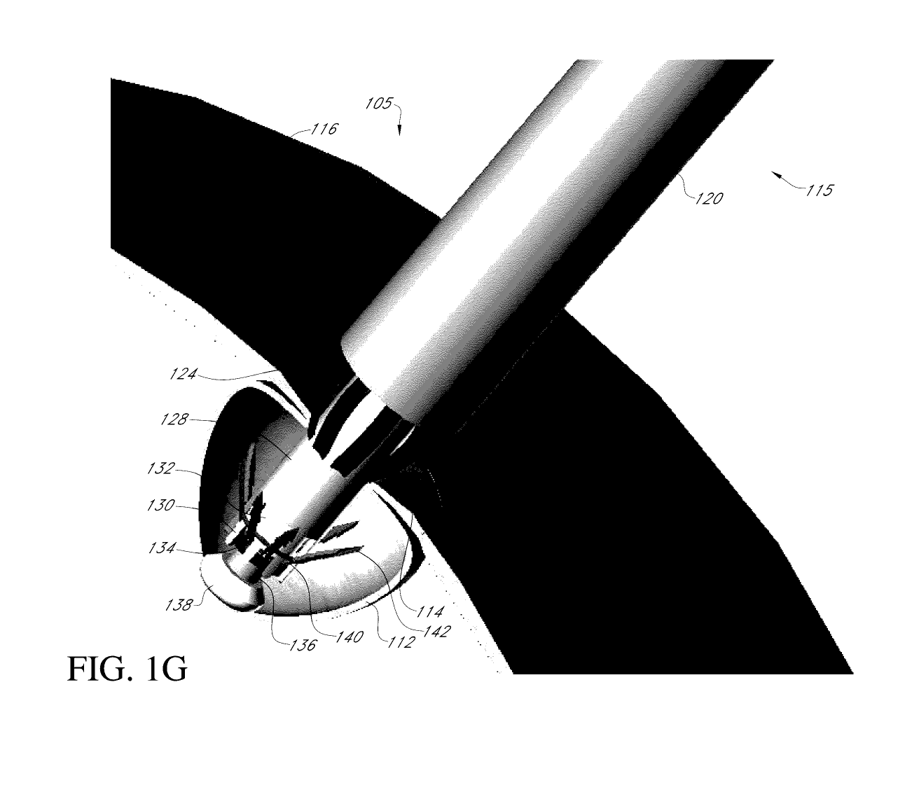

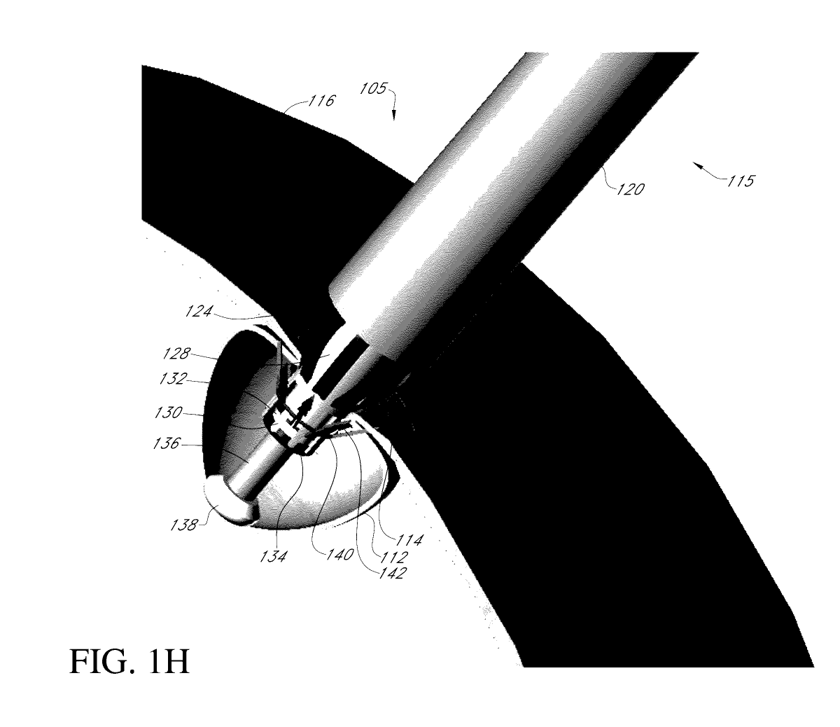

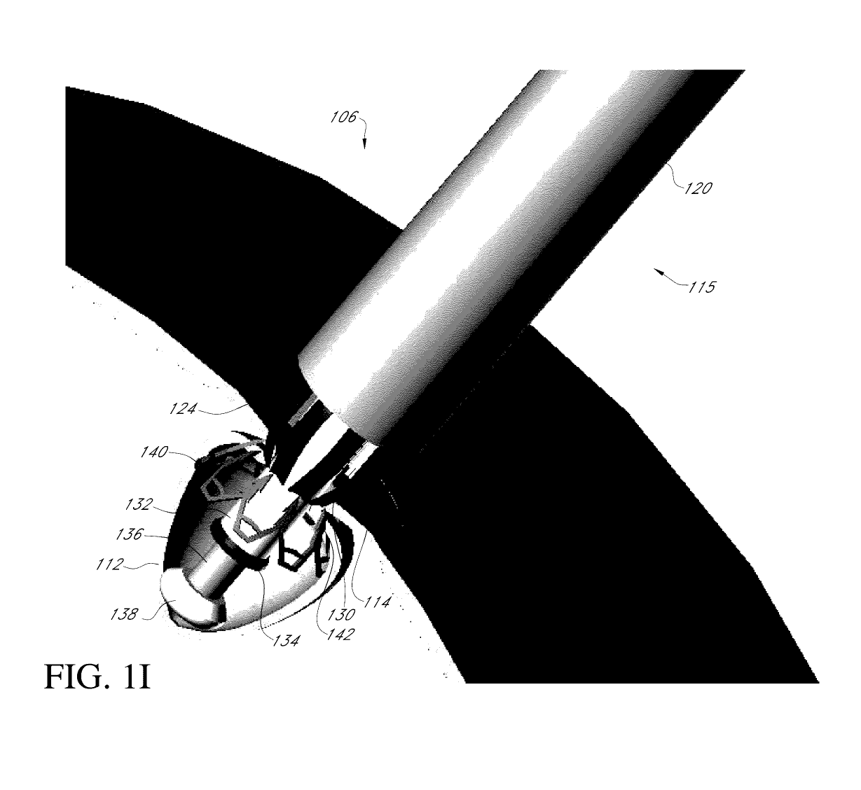

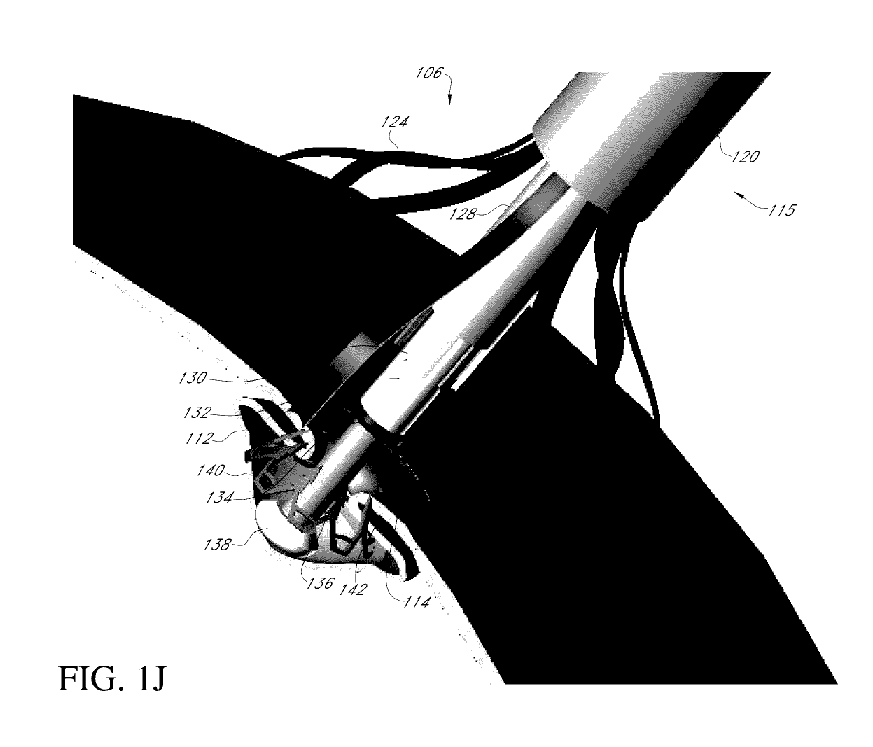

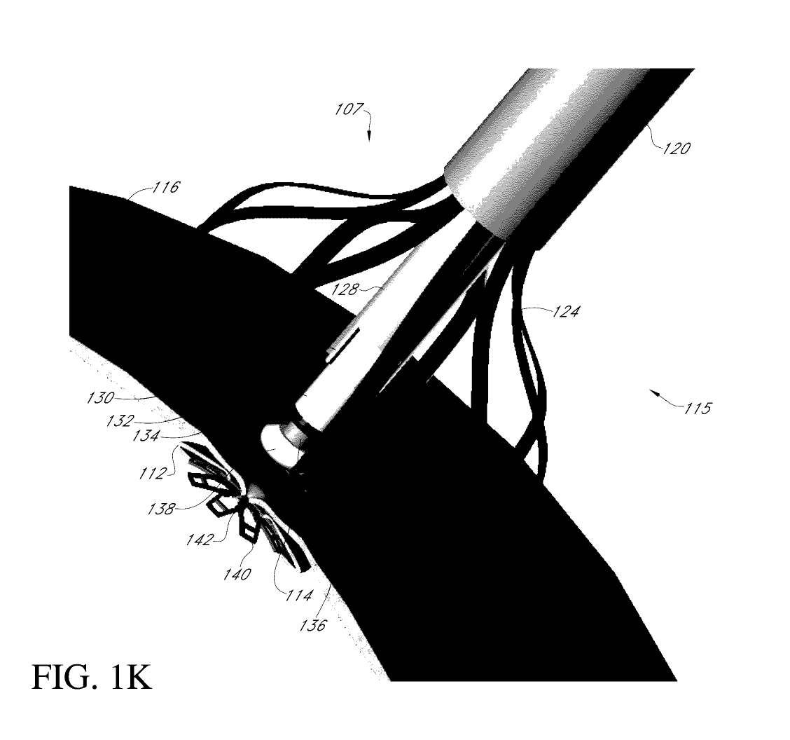

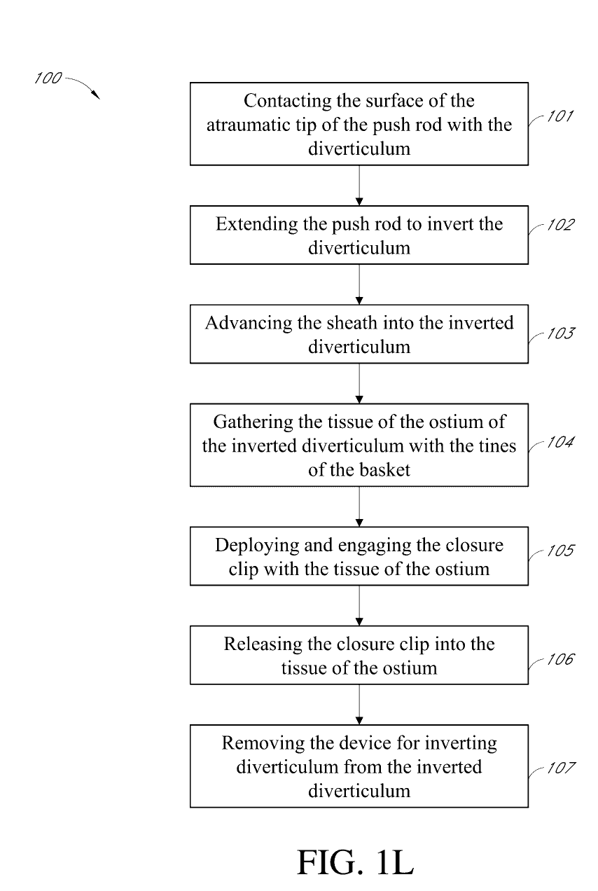

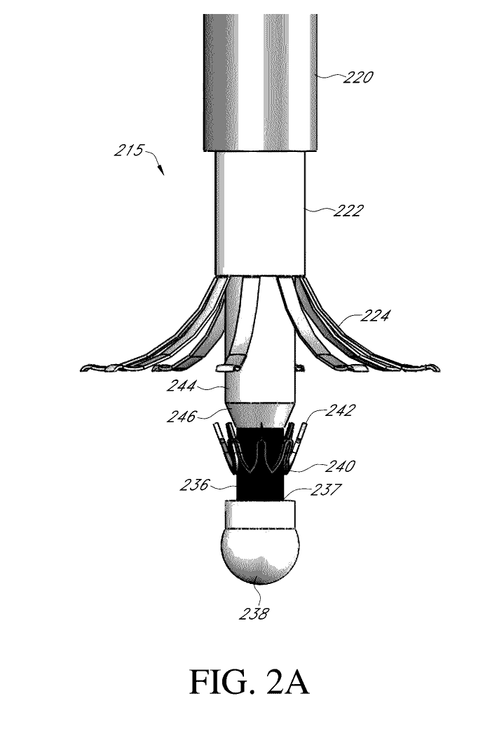

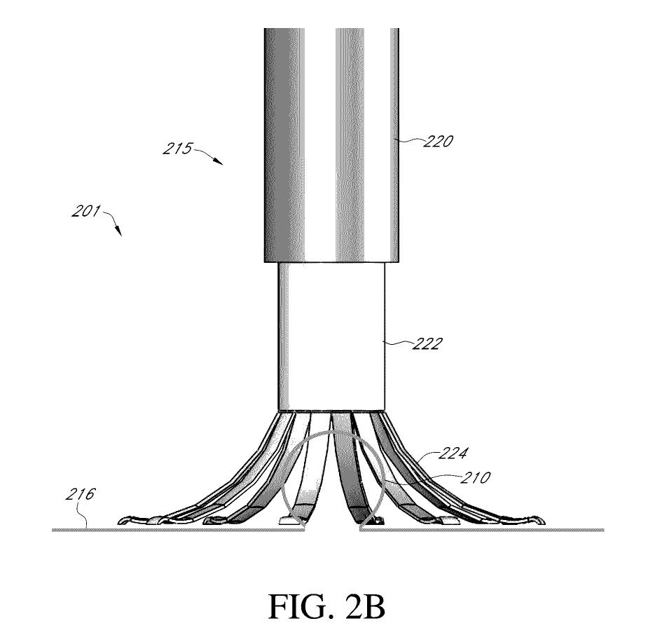

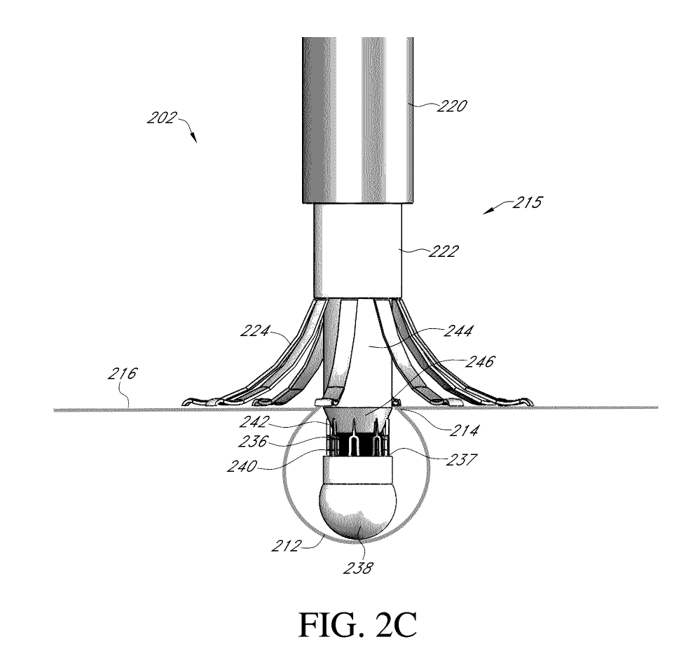

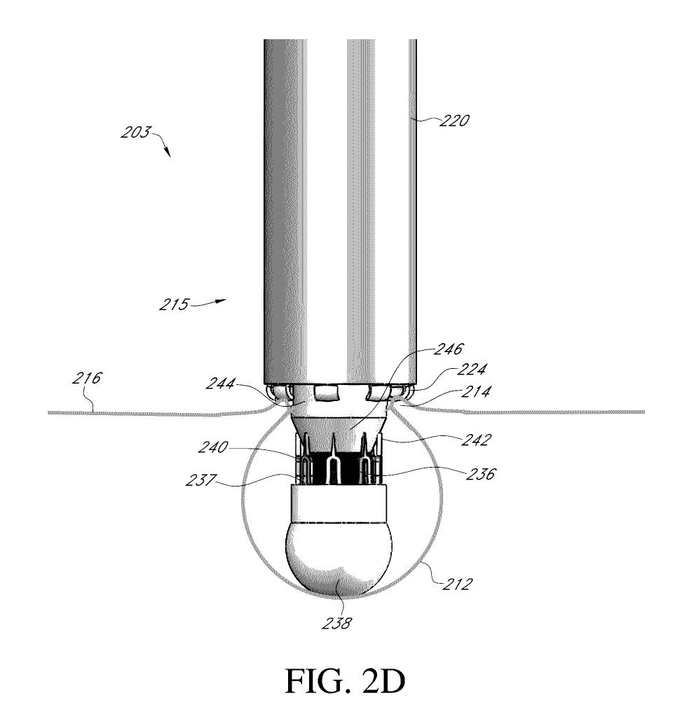

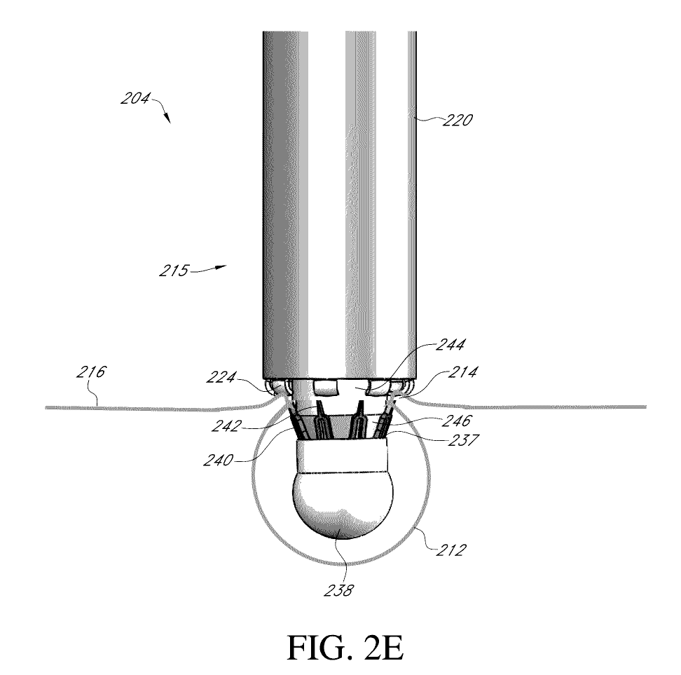

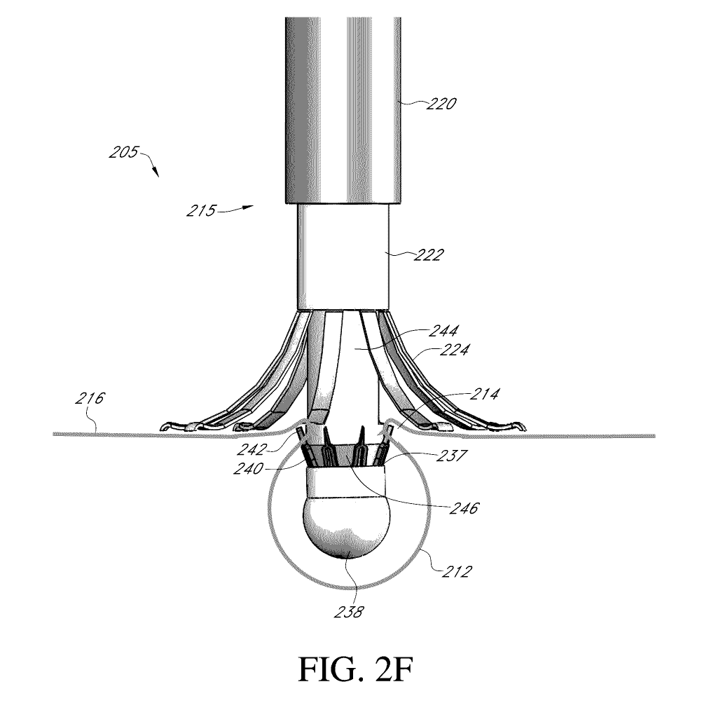

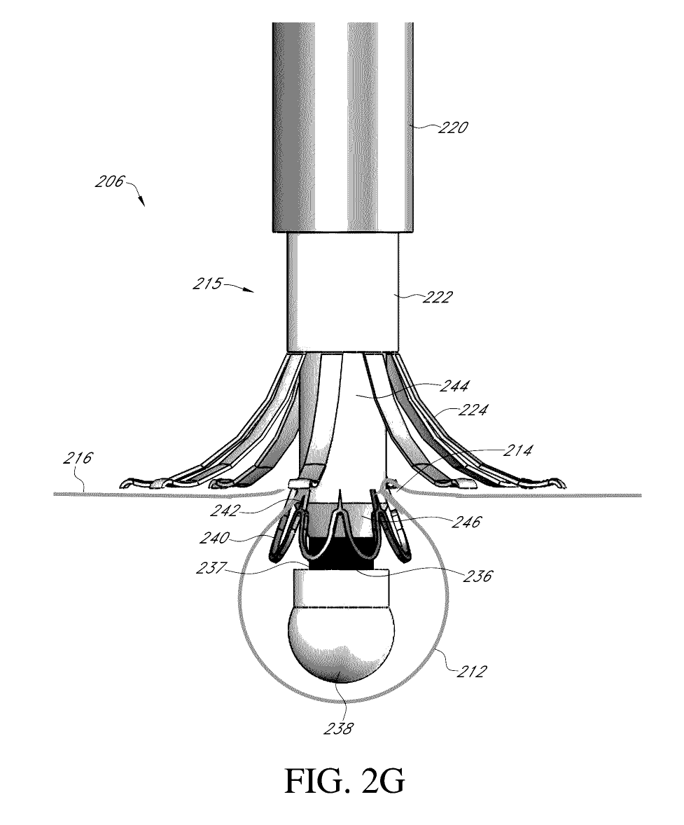

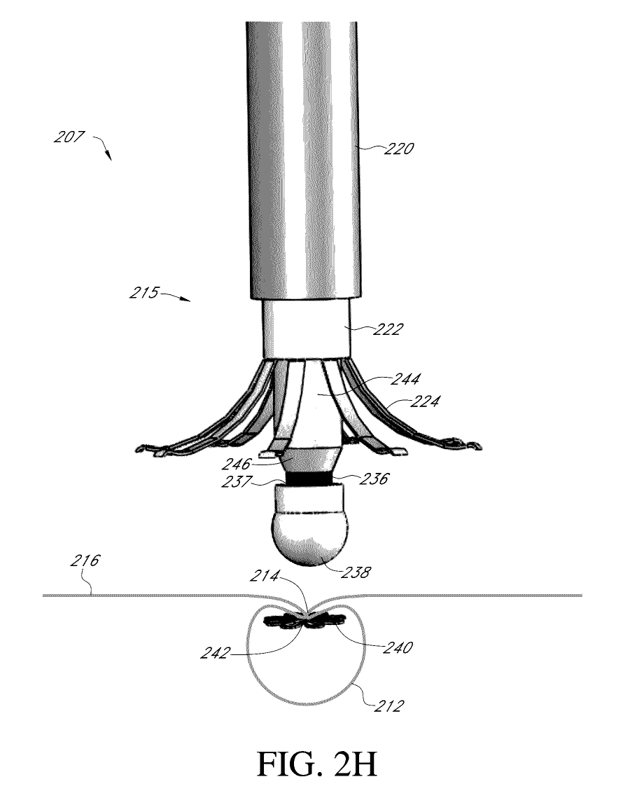

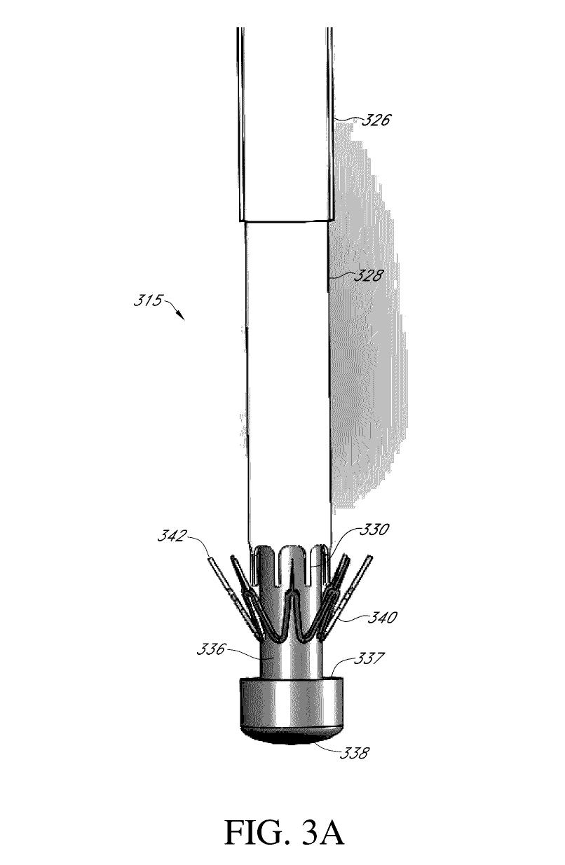

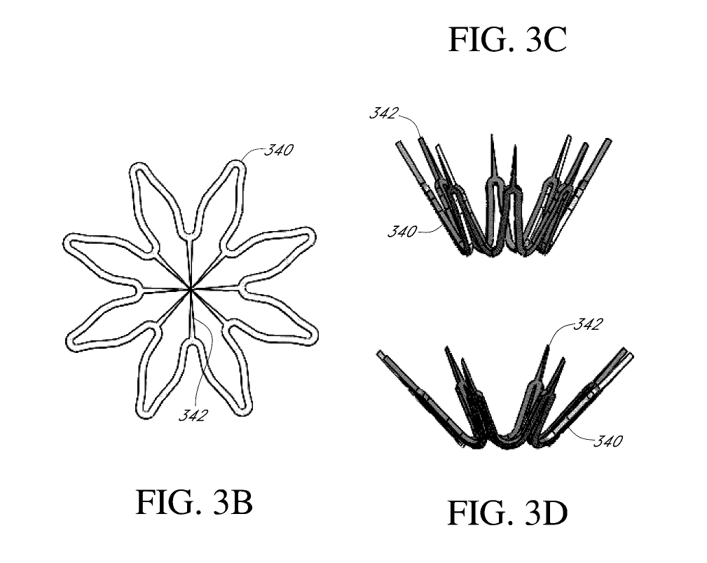

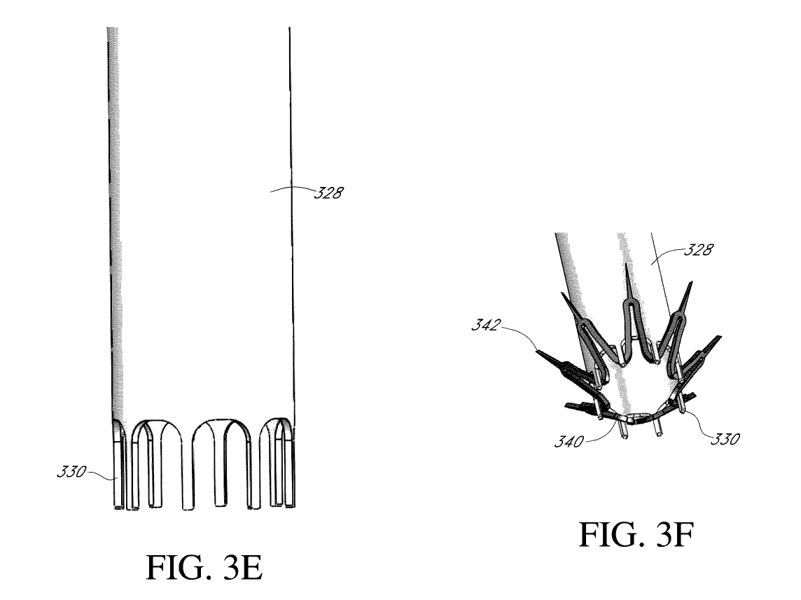

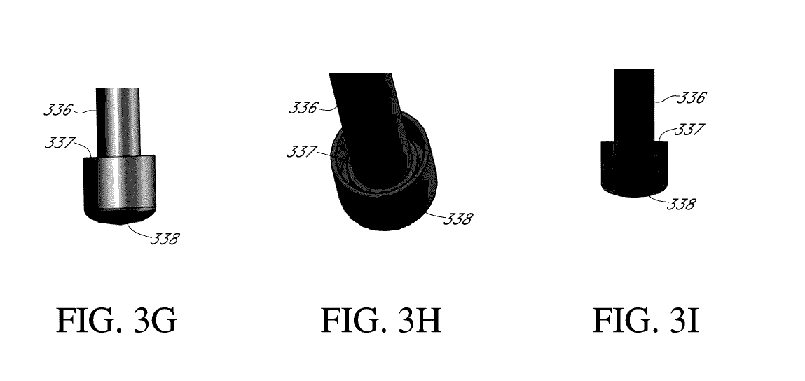

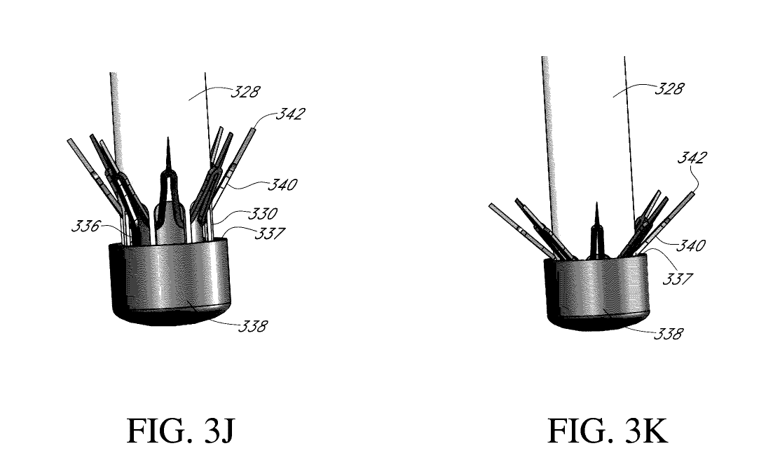

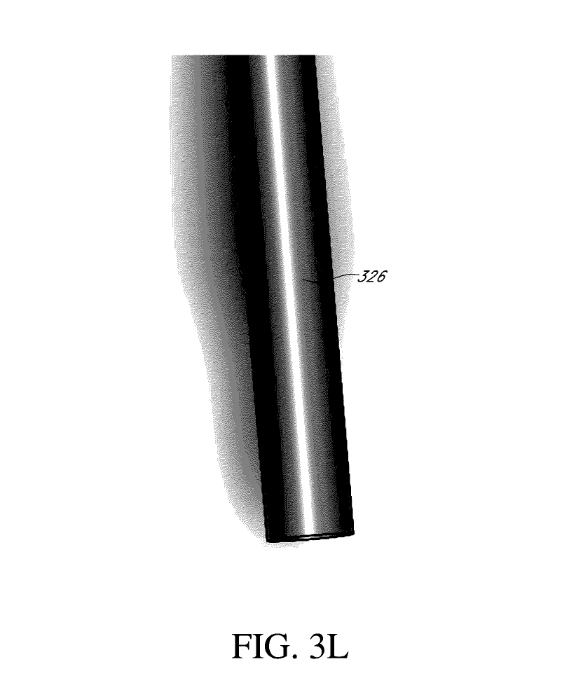

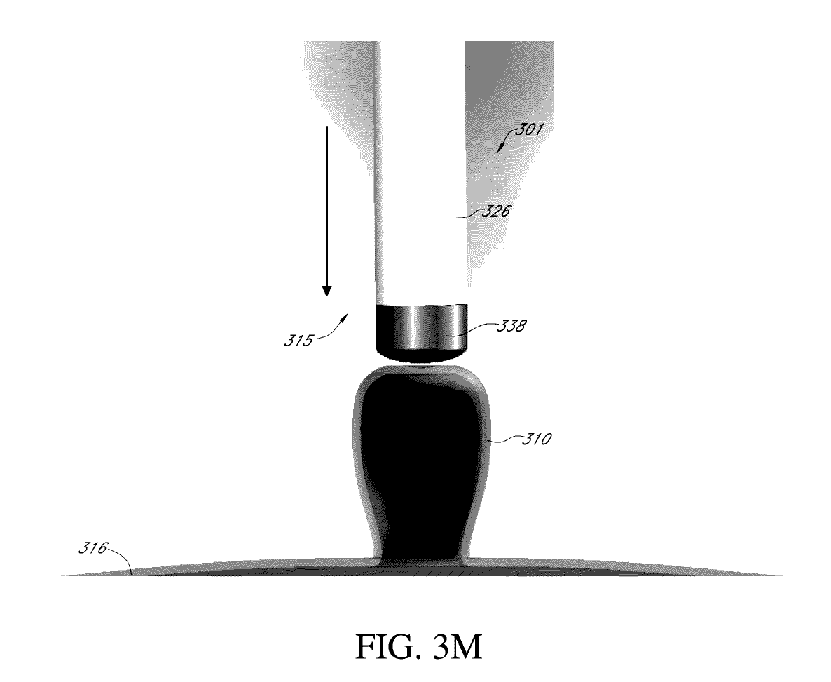

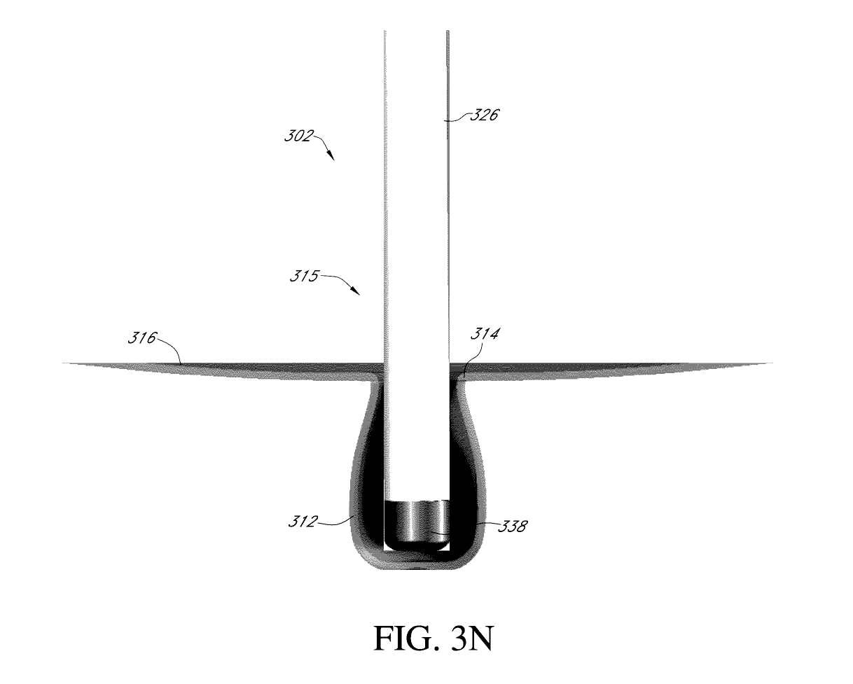

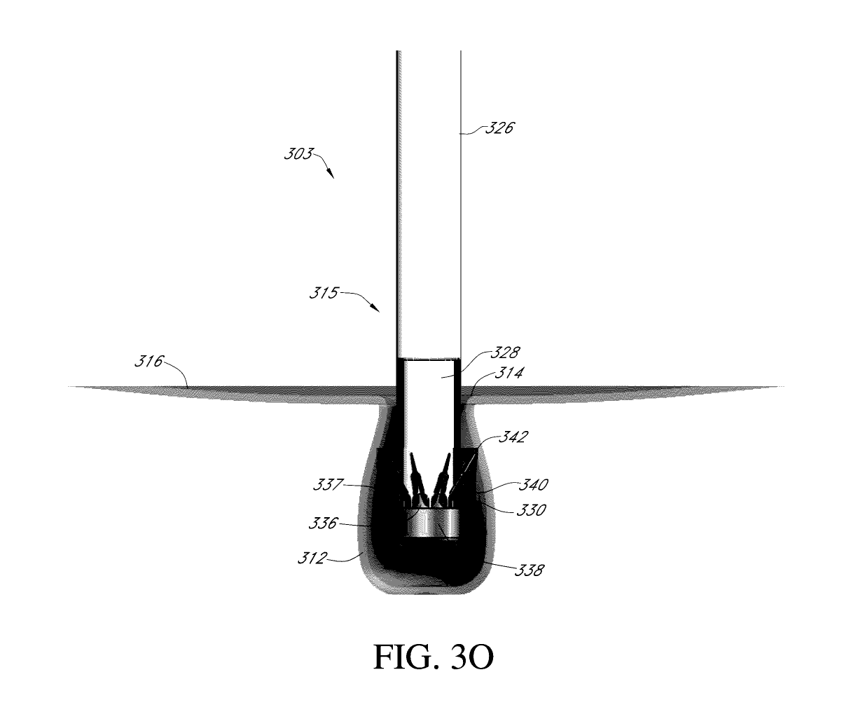

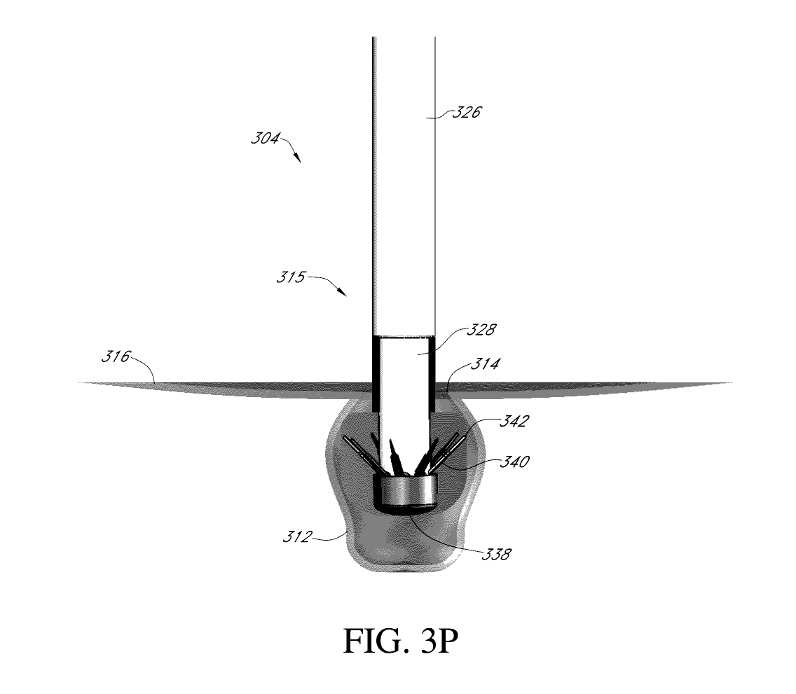

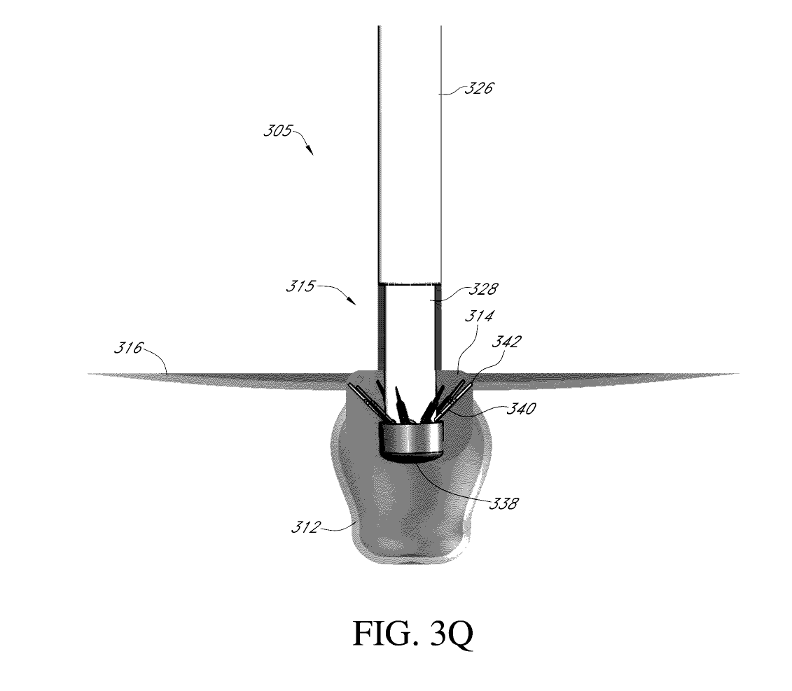

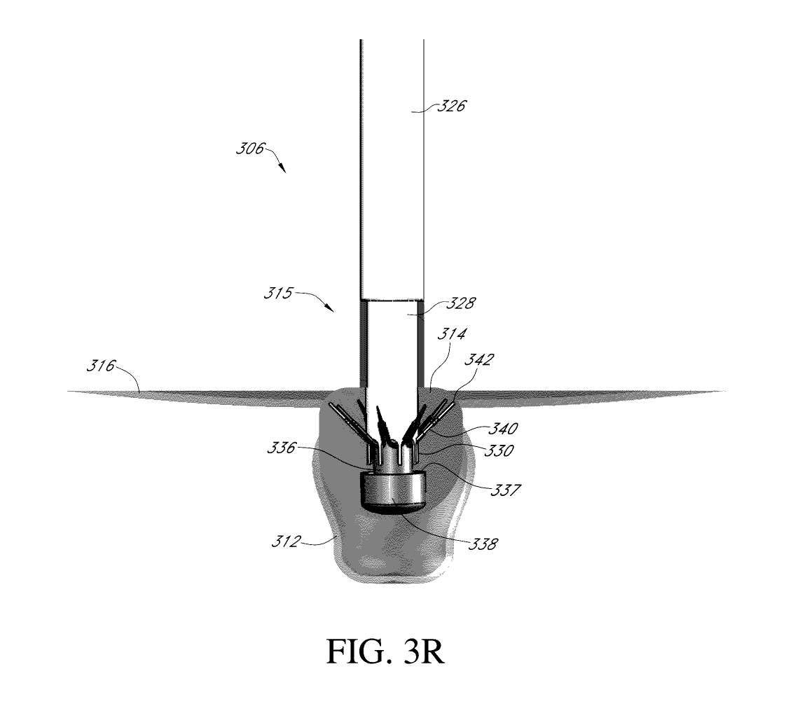

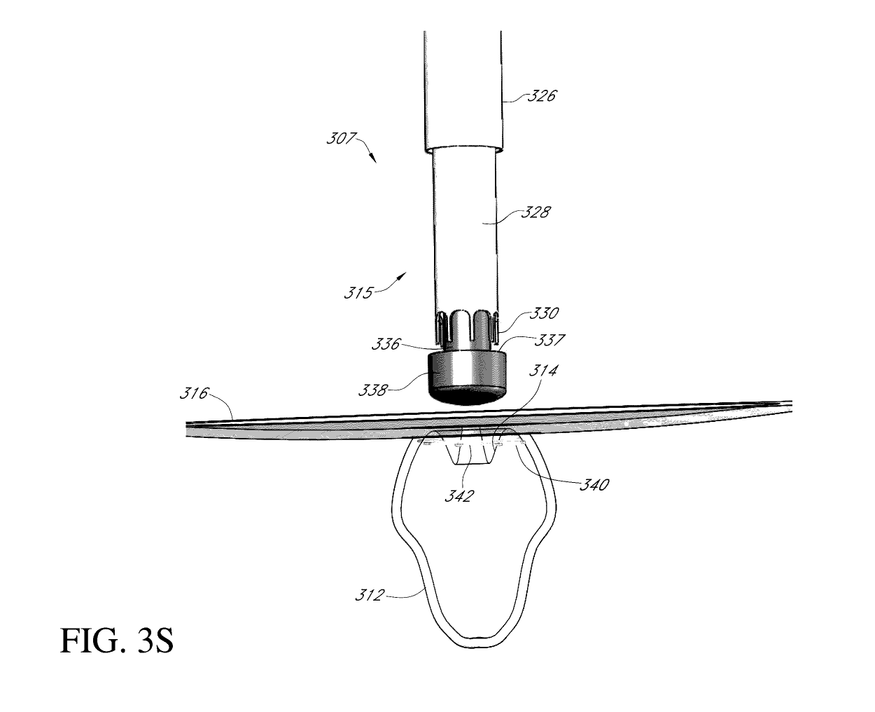

Disclosed herein are methods and devices for treating diverticular disease. FIGS. 1A-G, 2A-H, and 3A-R illustrate a plurality of views of a device for inverting diverticulum in a method for treating diverticulum disease. FIGS. 1A-G illustrate a plurality of views of an embodiments of a device for inverting diverticulum 115 in an embodiment of a method for inverting diverticula 100. FIGS. 2A-H illustrate a plurality of views of another embodiment of a device for inverting diverticulum 215 in some embodiments of a method of inverting diverticula. Finally, FIGS. 3A-R illustrate a plurality of view of another embodiment of a device for inverting diverticulum 315 in some embodiments of a method of inverting diverticula.

Turning first to the embodiment of a device for inverting diverticulum 115 illustrated in FIG. 1A, in some embodiments the device for inverting diverticulum 115 is composed of a plurality of components that are disposed coaxially about each other. As will be illustrated in the method of inverting diverticula 100, each of the components of the device for inverting diverticulum 115 are configured to be moveable relative to each other.

In some embodiments the device for inverting diverticulum 115 can include a closer 120, a basket shaft 122, a sheath 126, a clip tube 128, a tube stop 132, and a push rod 136. In some embodiments, the basket shaft 122 can further include a basket 124 at the distal end. As well, in some examples, the clip tube 128 can include a section including locking structures 130 at the distal end. As will be discussed, the locking structures 130 of the clip tube 128 can help to retain a closure clip 140. In some embodiments, the push rod 136 can further include a atraumatic tip 138 at the distal end. Each of the tubular components of the device for inverting diverticulum 115 can be composed of a hardened steel.

As seen in FIG. 1A, the push rod 136 is located at the center of the device for inverting diverticulum 115. In some embodiments, the push rod 136 includes an atraumatic tip 138 at the distal end. As will be discussed below, the atraumatic tip 138 of the push rod 136 can serve to invert a target diverticulum during the method of inverting diverticula 100.

In some examples, a tube stop 132 is disposed coaxially about the push rod 136. In some variants, a flange 134 is located at the distal end of the tube stop 132 such that the flange 134 forms a wider diameter than the distal opening of the tube stop 132. The push rod 136 and the tube stop 132 can move relative to each other such that the push rod 136 can be withdrawn into the tube stop 132. In some examples, the atraumatic tip 138 has a sufficiently wide diameter such that it prevents the push rod 136 from being withdrawn entirely into the tube stop 132. As will be discussed in more detail below, the tube stop 132—in particular the flange 134 of the tube stop 132—can help to adjust the angle on which the closure clip 140 is retained on the clip tube 128 as well as adjust the angle that the closure clip 140 penetrates into the target diverticulum.

In some variants, a clip tube 128 is disposed about the tube stop 132. As noted above, in some examples, the clip tube 128 includes a plurality of locking structures 130 that are located at the distal end of the clip tube 128. The locking structures 130 can be configured to retain a closure clip 140 on the distal end of the device for inverting diverticulum 115. The clip tube 128 is configured such that it is moveable relative to the tube stop 132. In some examples, the locking structures 130 of the clip tube 128 can extend past the flange 134 of the tube stop 132. As will be discussed below, the locking structures 130 can help to retain the closure clip 140 on the device for inverting diverticulum 115. In some variants, along with the flange 134, the locking structures 130 can adjust the angle of the closure clip 140 on the distal end of the device for inverting diverticulum 115 to better allow the closure clip 140 to engage with the tissue of the target inverted diverticulum. As well, the interaction of the flange 134 and the locking structures 130 can also allow the closure clip 140 to be deployed into the inverted diverticulum.

In some embodiments, a sheath 126 can be disposed about the clip tube 128. In some examples, the diameter of the sheath 126 should be wide enough such that it can accommodate the clip tube 128 and the attached closure clip 140. In some examples, the purpose of the sheath 126 is to prevent the closure clip 140 or the locking structures 130 of the clip tube 128 from catching onto anything prior to deployment or placed into the inverted diverticulum.

In some examples, the device for inverting diverticulum 115 can further include a basket shaft 122. As illustrated in FIG. 1A, in some variants, the basket shaft 122 can include a basket 124 located at the distal end of the basket shaft 122. As the basket shaft 122 is configured to be moveable relative to the sheath 126, the basket shaft 122 can be extended or withdrawn proximal and/or distal to the other components of the device for inverting diverticulum 115. As will be described in more detail below, the basket 124 is configured to retain the tissue about the ostium of the inverted diverticulum. This can help to better deploy the closure clip 140 into the target tissue.

Lastly, in some embodiments, the device for inverting diverticulum 115 can further include a closer 120. In some examples, the closer 120 is configured to be disposed about the internal components of the device for inverting diverticulum 115. As the closer 120 is moveable relative to the basket shaft 122, the closer 120 can extend distally to cover and retain the basket 124 of the basket shaft 122. In some examples this can help to maintain the minimal profile of the device for inverting diverticulum 115 prior to use of the device in the method of inverting diverticula 100.

As noted above, the various components of the device for inverting diverticulum 115 can be moveable relative to each other. As well, in order to maintain a minimal profile of the device for inverting diverticulum 115 prior to use of the device for inverting diverticulum 115 in the method of inverting diverticula 100, the closer 120 can be used to retain the basket 124 of the basket shaft 122. Similarly, to prevent the inadvertent deployment or interaction of the closure clip 140 with the surrounding environment, the sheath 126 can be disposed about the clip tube 128, tube stop 132, and the closure clip 140 retained in between. FIG. 1B illustrates the initial configuration of the device for inverting diverticulum 115 after the basket 124 is released from the closer 120. As described, the sheath 126 is disposed about the clip tube 128, tube stop 132, and the closure clip 140.

Once the device for inverting diverticulum 115 has been inserted into the body, the device can be used to treat a diverticulum. FIG. 1L illustrates a flow chart of the method of inverting diverticula 100. Each of the steps of the method of inverting diverticula 100 are illustrated in FIGS. 1C-K. In the method of inverting diverticula 100, the device for inverting diverticulum 115 is used to treat a diverticulum 110 that is located on the surface of the outer wall of colon 116

FIG. 1C illustrates step 101 in the method of inverting diverticula 100. As illustrated, the outer wall of colon 116 includes a diverticulum 110 that protrudes from the diverticulum 110. In some embodiments, in step 101, the atraumatic tip 138 of the push rod 136 is extended to contact the top surface of the diverticulum 110. In some examples, the atraumatic tip 138 enters substantially perpendicular to the colon surface. Initial contact adjustments may be necessary in order to evenly contact the diverticulum 110.

Next, as illustrated in FIG. 1D, the diverticulum 110 is inverted at step 102. In some embodiments, as can be seen, the push rod 136 can extend distally such that the atraumatic tip 138 compresses the diverticulum 110 to force it to invert. As the diverticulum 110 inverts, the outer lip of the tissue of the diverticulum 110 can form an ostium that can provide the closure clip 140 with tissue to engage with.

Once the diverticulum 110 has been inverted, as illustrated in FIG. 1E, step 103 involves fully forming the inverted diverticulum 112 and preparing the basket 124 to engage with the surrounding tissue of the ostium 114. As can be seen, after the diverticulum 110 is inverted with the atraumatic tip 138 of the push rod 136, the sheath 126 with the retained clip tube 128, tube stop 132, and closure clip 140 can be further advanced into the inverted diverticulum 112. The advancing of the device into the inverted diverticulum 112 better allows the tines of the basket 124 to engage with the ostium tissue 114 in tension (not pictured in FIG. 1E). In some examples, prior to the basket 124 touching the tissue of the ostium 114, the diameter of the basket 124 can be adjusted to 2-3 mm greater than the ostium 114. In some embodiments, once the basket 124 engages with the ostium 114 tissue, the outer wall of colon 116 is depressed by approximately 1 inch.

Next, FIG. 1F illustrates step 104 which describes tissue acquisition by the basket 124 once the device for inverting diverticulum 115 is in position. As illustrated, while the tissue of the inverted diverticulum 112 and ostium 114 are still under tension by the inserted distal end of the device for inverting diverticulum 115, the basket 124 is closed about the ostium 114 to draw up the tissue evenly. In some embodiments, this is accomplished by advancing the closer 120 distally past the tines of the basket 124. As the closer 120 is advanced, the tines of the basket 124 are drawn together to capture the tissue of the ostium 114. In some embodiments, if the tines of the basket 124 slip or the creases of the captured tissue are largely asymmetrical, the basket 124 can be opened and step 104 can be repeated.

Once the tissue of the ostium 114 has been captured by the basket 124, step 105 as illustrated in FIGS. 1G-H disclose deploying and engaging of the closure clip 140 with the tissue of the ostium 114 while located in the inverted diverticulum 112. As illustrated in FIG. 1G, the inverted diverticulum 112 can be expanded (e.g. with gas) to allow the closure clip 140 to expand. As described above, the attached closure clip 140 can be expanded to allow the clip tines 142 to flare outwards by withdrawing the sheath 126 in a proximal direction. In some embodiments, the withdrawn sheath 126 allows the clip tines 142 of the closure clip 140 to flare outwards. In some embodiments, as illustrated in FIG. 1H, the clip tube 128 and the tube stop 132 are withdrawn proximally to engage with the tissue of the inverted diverticulum 112 and ostium 114. As discussed, in some examples, because the inverted diverticulum 112 is expanded with gas, this prevents the inverted diverticulum 112 tissue from bunching up over the tines. As illustrated, the clip tines 142 of the closure clip 140 are targeted to penetrate into the outer wall of colon 116.

In some examples, once the closure clip 140 has penetrated into the tissue, the method of inverting diverticula 100 can further include step 106 which illustrates the releasing of the closure clip 140 into the tissue. As illustrated in FIG. 1I, the inverted diverticulum 112 is longer inflated (e.g. the gas can be stopped). To release the closure clip 140 into the target tissue, the clip tube 128 can first be withdrawn proximally. In some examples, the proximal withdrawing of the locking structures 130 releases the closure clip 140 from the angle on the clip tube 128 and allows the closure clip 140 to begin to rotate into its final position. The tube stop 132 is not withdrawn so as to maintain the closure clip 140 on the device. Next, as illustrated in FIG. 1J, after the clip has rotated into its final position (e.g. planar position), the tube stop 132 is withdrawn in a proximal direction. In some examples, the tube stop 132 and the flange 134 slips through the center of the inserted closure clip 140. As well, as illustrated in FIG. 1J, the basket 124 can be opened to release the tissue of the ostium 114. In some embodiments (not pictured), the closure clip 140 can be sprung to the clip tube 128 such that the withdrawal of the tube stop 132 and flange 134 can be self-driven.

Finally, the device for inverting diverticulum 115 can be removed from the inverted diverticulum 112 in step 107 as illustrated in FIG. 1K. To remove the device for inverting diverticulum 115 from the inverted diverticulum 112, the push rod 136 and atraumatic tip 138 can be withdrawn gently to pull it past the implanted closure clip 140. As seen in FIG. 1K, once the push rod 136 and atraumatic tip 138 are removed from the inverted diverticulum 112, the closure clip 140 is allowed to rotate such that it is fully flattened. In some examples, as the closure clip 140 flattens, the clip tines 142 of the closure clip 140 capture the tissue of the ostium 114 so as to flatten and secure the inverted diverticulum 112 closed on the outer wall of colon 116.

FIGS. 2A-H illustrates another embodiment of the device for inverting diverticulum 215. FIG. 2A illustrates a side perspective of the distal end of the device for inverting diverticulum 215.

Turning now to another embodiment of a device for inverting diverticulum 215, in some embodiments the device for inverting diverticulum 215 is composed of a plurality of components that are disposed coaxially about each other. Similar to the method of inverting diverticula 100, in the method of inverting diverticula as illustrated in FIGS. 2B-H, each of the components of the device for inverting diverticulum 215 are configured to be moveable relative to each other.

In some embodiments, the device for inverting diverticulum 215 can include a closer 220, a basket shaft 222, a ramp tube 244, and a push rod 236. As can be seen, the device for inverting diverticulum 215 is largely similar to the device for inverting diverticulum 115 with a few adjustments. For example, in some embodiments, the device for inverting diverticulum 215 includes a ramp tube 244 that can further include a ramped portion 246 at the distal end of ramp tube 244. Unlike the closure clip 140 in the device for inverting diverticulum 115 that is attached to the locking structures 130 of the clip tube 128, the closure clip 240 is disposed about the push rod 236. In some embodiments, the atraumatic tip 238 of the device for inverting diverticulum 215 can further include an inner opening 237. Each of the tubular components of the device for inverting diverticulum 115 can be composed of a hardened steel.

As illustrated in FIG. 2A, the push rod 236 is located at the center of the device for inverting diverticulum 215. In some embodiments, the push rod 236 includes an atraumatic tip 238 at the distal end. In some examples, at the connection point between the push rod 236 and the atraumatic tip 238, the outer surface of the push rod 236 and the inner surface of the atraumatic tip 238 can form an inner opening 237. In some examples, the inner opening 237 can be configured to accommodate a portion of the closure clip 240 to adjust the angle that the closure clip 240 is located on the push rod 236. In other embodiments, the inner opening 237 of the atraumatic tip 238 can help to keep the closure clip 240 in a “safe” position prior to delivery. As will be discussed below, when it is ready to deliver the closure clip 240, the atraumatic tip 238 can be withdrawn to guide the closure clip 240 into the ramped position.

In some examples, the ramp tube 244 is disposed coaxially about the push rod 236. In some variants, the ramp tube 244 includes a ramped portion 246 that is located at the distal end of the ramped portion 246. The ramp tube 244 can be moveable relative to the push rod 236. In some examples, the ramped portion 246 of the ramp tube 244 can move an attached closure clip 240 into the inner opening 237 of the atraumatic tip 238 to alter the angle of the closure clip 240. The ramped portion 246 can help to adjust the angle on which the closure clip 240 is retained on the push rod 236 as well as to adjust the angle that the closure clip 240 penetrates into the target diverticulum. The ramped portion 246 can flare the clip into delivery position.

In some variants, the basket shaft 222 can be disposed about the ramp tube 244. In some examples, the basket shaft 222 can include a basket 224 located at the distal end of the basket shaft 222. As the basket shaft 222 is configured to be moveable relative to the ramp tube 244, the basket shaft 222 can be extended or withdrawn proximal and/or distal to the other components of the device for inverting the diverticulum 215. As will be described in more detail below, the basket 224 can be configured to retain the tissue about the ostium of the inverted diverticulum. This can help to better deploy the closure clip 240 into the target tissue.

In some embodiments, the device for inverting diverticulum 215 can further include a closer 220. In some examples, the closer 220 is configured to be disposed about the internal components of the device for inverting diverticulum 215. As the closer 220 is moveable relative to the basket shaft 222, the closer 220 can extend distally to cover and retain the basket 224 of the basket shaft 222. In some examples, this can help to maintain the minimal profile of the device for inverting diverticulum 215 prior to use of the device in the method of inverting diverticula.

The device for inverting diverticulum 215 can be used to treat a diverticulum. FIGS. 2B-H illustrate one embodiment of the method of inverting diverticula. The method of inverting diverticula is generally similar to the method of inverting diverticula 100.

FIG. 2B illustrates step 201 in the method of inverting diverticula. As illustrated, in some embodiments, after the device for inverting diverticulum 215 is inserted through the trocar, the closer 220 can be withdrawn in a proximal direction to release the 22 and the basket 224. The device for inverting diverticulum 215 can then be advanced until the basket 224 is centered around the target diverticulum 210.

Next, at FIG. 2C, the method of inverting diverticula can include step 202 wherein the atraumatic tip 238 on the push rod 236 can be advanced in a distal direction to invert the diverticulum 210. In some embodiments, the distal end of the atraumatic tip 238 can be spring loaded to prevent excessive force from being placed on the diverticulum 210 and in order to accommodate diverticulum 210 of different sizes. In some examples, at step 202, the closure clip 240 is retracted into the inner opening 237 of the atraumatic tip 238. In this configuration, the clip tines 242 is in a “safe” position, such that the clip tines 242 do not interfere with any tissue as it is inserted into the inverted diverticulum 112. In some examples, the inverted diverticulum 212 can be inflated (e.g. with gas) to allow better presentation of the ostium 214 to the closure clip 240.

In some embodiments, the method of inverting diverticula can include step 203 illustrated in FIG. 2D. Here, the basket 224 of the basket shaft 222 is closed around the tissue of the ostium 214 formed from the inverted diverticulum 212 on the outer wall of colon 216. In some examples, the basket 224 can grab the tissue by advancing the closer 220 in a distal direction until it completely covers the length of the basket 224. This can allow the basket 224 to pull and secure the healthy tissue of the ostium 214 around the ramp tube 244.

As illustrated in FIG. 2E, the method of inverting diverticula can include step 204 wherein the closure clip 240 is ramped into a “deploy” position. In some examples, at step 204, the push rod 236 is withdrawn in a distal direction with respect to the ramp tube 244. The ramped portion 246 of the ramp tube 244 can guide the closure clip 240 into the “deploy” position. In some examples, in the “deploy” position, the clip tines 242 of the closure clip 240 are flared outwards and engage with the gathered tissue at the ostium 214 of the inverted diverticulum 212. In some embodiments (not illustrated) the closure clip 240 can be released by continuing to withdraw the atraumatic tip 238 in a proximal direction. While the inner ledge 237 holds the clip in place, the movement of the atraumatic tip 238 is under the clip and ejects the closure clip 240 into the tissue as the closure clip 240 is moved along the ramped portion 246 of the ramp tube 244. Once the closure clip 240 is free of the inner ledge 237, it can begin to expand outwardly to return to its original planar shape.

Once the clip tines 242 of the closure clip 240 has been inserted into the tissue of the ostium 214, the method of inverting diverticula can proceed to step 205. As illustrated in FIG. 2F, the closer 220 can be withdrawn in a distal direction. As the closer 220 is withdrawn, the basket 224 can be opened—thereby releasing the gathered tissue of the ostium 214 around the neck of the inverted diverticulum 212.

In some embodiments, the method of inverting diverticula 200 can then include step 206 as illustrated in FIG. 2G. Once the clip tines 242 of the closure clip 240 are secured in the tissue of the ostium 214, the push rod 136 can be advanced in a distal direction. In doing so, the closure clip 240 is released from the inner opening 237 of the atraumatic tip 238. This can allow the closure clip 240 to rotate with the clip tines 242 in gripping tissue around the ramp tube 244.

Once the closure clip 240 has been released from the inner opening 237 of the atraumatic tip 238, the method of inverting diverticula can proceed to step 207 where the device for inverting diverticulum 215 is retracted from the inverted diverticulum 112. In some embodiments, once the closure clip 240 has been released, the ramp tube 244, the ramped portion 246 of the ramp tube 244, the push rod 236, and the atraumatic tip 238 of the push rod 236 can be retracted through the ostium 214 of the inverted diverticulum 112. In some examples, once the distal end of the device for inverting diverticulum 215 has been fully retracted, the closure clip 240 can closed to its natural flat shape and grip the healthy tissue of the ostium 214 together with the clip tines 242.

Finally, FIGS. 3A-S illustrate another embodiment of the device for inverting diverticulum 315. FIG. 3A illustrates a side perspective of the distal end of the device for inverting diverticulum 315. FIGS. 3B-L illustrate a plurality of views of the components in the device for inverting diverticulum 315. Many of the illustrations of the components of the device for inverting diverticulum 315 can be similarly applicable to the device for inverting diverticulum 115 and device for inverting diverticulum 215 discussed above.

The device for inverting diverticulum 315 has elements that resemble or are similar to the device for inverting diverticulum 115 and device for inverting diverticulum 215 described above. Accordingly, numerals used to identify features of the device for inverting diverticulum 115 and device for inverting diverticulum 215 are incremented by a factor of one hundred to identify like features of the device for inverting diverticulum 315. This numbering conventional generally applies to the remainder of the figures. Any component or step disclosed in any embodiment in this specification can be used in other embodiments.

In some embodiments, the device for inverting diverticulum 315 (as illustrated in FIG. 3A), can be composed of a plurality of components that are disposed coaxially about each other. As will be illustrated in the method of inverting diverticula, each of the components of the device for inverting diverticulum 315 are configured to be moveable relative to each other. Unlike the previous two embodiments, in some examples, the device for inverting diverticulum 315 does not include a basket for securing the ostium of the inverted diverticulum. As discussed above, in some embodiments, each of the tubular components of the device for inverting diverticulum 115 can be composed of a hardened steel.

As seen in FIG. 3A, the push rod 336 is located at the center of the device for inverting diverticulum 315. As can be seen, in some embodiments, the closure clip 340 is disposed about the push rod 336 of the device. In some embodiments, the push rod 336 can include an atraumatic tip 338 at the distal end.

FIGS. 3B-D illustrate a plurality of views of the closure clip 340. Each of these illustrations can be applicable to any discussion of the closure clip provided above. FIG. 3B illustrates a top view of an embodiment of the closure clip 340 in its relaxed state. In its relaxed state, the closure clip 340 is flat with the clip tines 342 pointing inward. In some embodiments, this is the form the closure clip 340 will take after it has been delivered inside of the inverted diverticulum 112. In some embodiments, the closure clip 340 can have a tines that are 1.88 mm. FIG. 3C illustrates the closure clip 340 as it is loaded on the locking structures 330 of the clip tube 328. In this loaded state, the closure clip 340 is in a semi-flared shape. Lastly, FIG. 3D illustrates the closure clip 340 as it is flared even more for delivery to capture the tissue in the ostium 314. As was discussed, and will be discussed below, in the various embodiments of the method of inverting diverticula, the flaring of the clip tines 342 provide the closure clip 340 with a broader reach to engage the surrounding tissue of the ostium 314.

In some examples, at the connection point between the push rod 336 and the atraumatic tip 338, the outer surface of the push rod 336 and the inner surface of the atraumatic tip 338 can form an inner opening 337. In some examples, the inner opening 337 can be configured to accommodate a portion of the closure clip 340 to adjust the angle that the closure clip 340 is located on the push rod 336. FIGS. 3G-I illustrate a side, top, and cross-sectional view of the atraumatic tip 338. As is illustrated, the inner opening 337 provided between the outer surface of the push rod 336 and the inner surface of the atraumatic tip 338 provides an opening to accommodate a portion of the closure clip 340. As well, as illustrated in FIG. 3H, in some embodiments, the inner opening 337 can include a ledge that is adjacent the outer surface of the push rod 336.

In some examples, a clip tube 328 can be disposed about the push rod 336. In some embodiments, the clip tube 328 includes locking structures 330 at the distal end of the clip tube 328. As will be discussed in more detail below, the locking structures 330 can engage with the closure clip 340 that is disposed about the push rod 336 to secure the closure clip 340 on the distal end of the device for inverting diverticulum 315. As noted above, because the clip tube 328 and the push rod 336 are moveable relative to each other, withdrawing or advancing the clip tube 328 can cause the locking structures 330 to interact with the closure clip 340 to alter the angle in which the clip tines 342 are flared on the distal end of the device.

FIGS. 3E-F illustrate the clip tube 328 and a bottom perspective view of the clip tube 328 with closure clip 340 engaged. As illustrated in FIG. 3E, the locking structures 330 can include a plurality of equally spaced structures. FIG. 3F illustrate the closure clip 340 as it is engaged with the locking structures 330. As seen, each apex of the closure clip 340 is linked around the locking structures 330 to create the loaded flare shape—wherein the clip tines 342 are flared outwards and pointed in a proximal direction.

FIGS. 3J-K provide an illustration of the interaction between the locking structures 330 of the clip tube 328, the closure clip 340, and the atraumatic tip 338 of the push rod 336. FIG. 3J illustrates the closure clip 340 as it is loaded on the clip tube 328. In some examples, as the atraumatic tip 338 is moved up towards the clip tube 328, the ledge in the inner opening 337 can bump the apices of the closure clip 340. This can push them upward to cause a larger flare. FIG. 3K illustrates the distal end of the device for inverting diverticulum 315 and the configuration of the closure clip 340 during delivery. The structure of the inner opening 337 and atraumatic tip 338 allows the closure clip 340 to be pulled into the tissue without being inverted due to the force.

Lastly, in some embodiments, the device for inverting diverticulum 315 can further include a sheath 326 that can be disposed about the clip tube 328. In some examples, as seen in FIG. 3L, the sheath 326 can be wide enough such that it can accommodate the clip tube 328 and the attached closure clip 340. In some examples, the purpose of the sheath 326 is to prevent the closure clip 340 or the locking structures 330 of the clip tube 328 from unintentionally interacting with any tissue prior to deployments of the closure clip 340. The sheath 326 therefore protects the surrounding tissue as the device for inverting diverticulum 315 is inserted. As will be seen, the sheath 326 can be removed prior to deployment.

As discussed above, the device for inverting diverticulum 315 can be used to treat a diverticulum. FIGS. 3M-S illustrates another embodiment of the method of inverting diverticula. However, as will be discussed below, unlike the method of inverting diverticula discussed above, the device for inverting diverticulum 315 in the method of inverting diverticula does not include a basket for capturing the tissue of an inverted diverticula.

FIG. 3M can illustrate step 301 in the method of inverting diverticula. As is illustrated, the outer wall of colon 316 can include a diverticulum 310 that protrudes from the surface of the colon. In some embodiments, the atraumatic tip 338 located at the distal end of the device for inverting diverticulum 315 can be used to manually invert the diverticulum 310.

FIG. 3N next illustrates step 302 which shows the distal end of the device for inverting diverticulum 315 located in the inverted diverticulum 312. The distal end of the atraumatic tip 338 can further push into the inverted diverticulum 312 such that the ostium 314 is disposed snugly about the sheath 326.

The method of inverting diverticula can then include step 303 as illustrated in FIG. 3O. In some embodiments, at step 303, the sheath 326 is withdrawn in a proximal direction such that the closure clip 340 is exposed within the inverted diverticulum 312. As discussed above, the closure clip 340 at this stage is in the configuration illustrated in FIG. 3C, wherein the closure clip 340 is in a semi-flared state. In some examples, the inverted diverticulum 312 can be inflated (e.g. with gas) to allow better presentation of the ostium 214 to the closure clip 340.

In some examples, as illustrated in FIG. 3P, the method of inverting diverticula can include step 304 wherein the push rod 336 and the atraumatic tip 338 is retracted in a distal direction. As the push rod 336 is retracted, the ledge located within the atraumatic tip 338 (not pictured) can pull the closure clip 340 in a proximal direction, causing the closure clip 340 to flare to a larger diameter. In some examples, the closure clip 340 can be in the configuration illustrated in FIG. 3D wherein the flaring of the clip tines 342 can provide the closure clip 340 with a broader reach in order to engage the surrounding tissue of the ostium 314.

Once the closure clip 340 is flared outwards, the method of inverting diverticula can then include step 305 wherein the entirety of the device for inverting diverticulum 315 is retracted in a proximal direction. In some embodiments, as illustrated in FIG. 3Q, the device for inverting diverticulum 315 can be retracted in a proximal direction, this can serve to seat the clip tines 342 of the closure clip 340 into the surrounding tissue of the ostium 314. In some examples, once the clip tines 342 are in the surrounding tissue of the ostium 314, the closure clip 340 is ready to be released.

In some examples, the method of inverting diverticula can then include step 306 in order to begin the step of releasing the closure clip 340 into the tissue of the ostium 314. As illustrated in FIG. 3R, the atraumatic tip 338 of the push rod 336 is advanced in a distal direction. This can relax the closure clip 340 from the flared configuration to a semi-flared configuration. In some examples, the advancing of the atraumatic tip 338 in the inverted diverticulum 112 can allow the closure clip 340 to be released from the locking structures 330 of the clip tube 328. In some embodiments, (not illustrated) the push rod 336 can include a ramped portion. The closure clip 340 can be released by continuing to withdraw the atraumatic tip 338 in a proximal direction to cause the closure clip 340 to be ejected from the push rod 336.

Once the closure clip 340 has been released from the locking structures 330 of the clip tube 328, the distal end of the device for inverting diverticulum 315 can be retracted from the inverted diverticulum 112. As illustrated in FIG. 3S, in some embodiments, the configuration of the closure clip 340 can allow the atraumatic tip 338 to be pulled through the center of the closure clip 340. As the distal end of the device for inverting diverticulum 315 is pulled from the ostium 314 of the inverted diverticulum 312, the closure clip 340 can close around the captured tissue of the ostium 314. In some examples, the closure clip 340 can take the form of the closure clip 340 illustrated in FIG. 3B.

As the clip is being deployed, frequently the inverted diverticulum is pressure collapsed from the differential insufflation pressure being greater in the bowel than in the abdomen. As a result, the tissue snugly conforms to the clip deployment mechanism and the closure clip that is placed in the inverted diverticulum illustrated above. This can result in inconsistent implantation of the clip tines of the closure clip into the colon tissue as a result of the clip tines being unable to engage with the tissue at an advantageous angle. Because of the differential of pressures in the bowel and the abdomen, the inverted diverticulum can collapse about the closure clip and cause the clip tines to slip rather than engage.

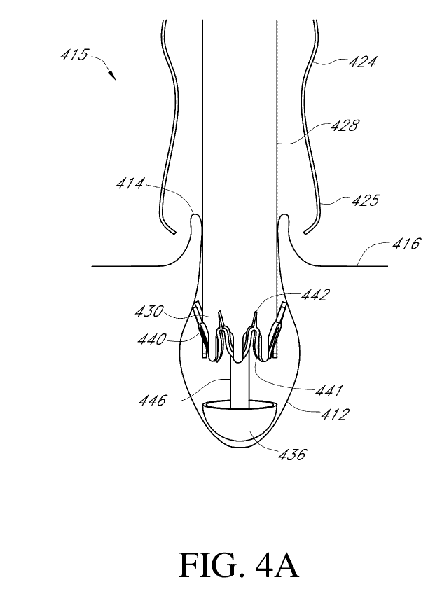

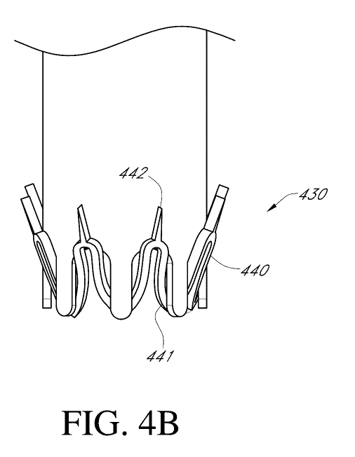

FIGS. 4A-B provide side views of an existing device for treating diverticular disease 415 used on a pressure collapsed inverted diverticulum 412. As can be seen in FIG. 4A, the device for treating diverticular disease 415 includes a basket 424 with a plurality of basket tines 425, a clip tube 428, a closure clip 440, and a push rod 446 with a atraumatic tip 436 at the distal end. FIG. 4B provides a detailed view of the distal end of locking structures 430. The distal end of locking structures 430 includes a plurality of rectangular castles. A closure clip 440 is attached to the distal end of the locking structures 430. The closure clip 440 includes a plurality of clip tines 442 and a plurality of engagement centers 441. The closure clip 440 is configured on the distal end of locking structures 430 such that each of the engagement centers 441 is disposed about each of the locking structures 430 and each of the clip tines 442 extend in a proximal direction. As discussed above, the pressurized colon puts pressure on the inverted diverticulum 412 and causes it to “collapse”—having a narrowed width. This narrowing makes the outer tissue wall of colon 416 of the ostium 414 difficult to approximate. This creates a poor incident angle for the clip tines 442 against the outer tissue wall of colon 416 of the ostium 414 and therefore makes it difficult for the clip tines 442 to penetrate.

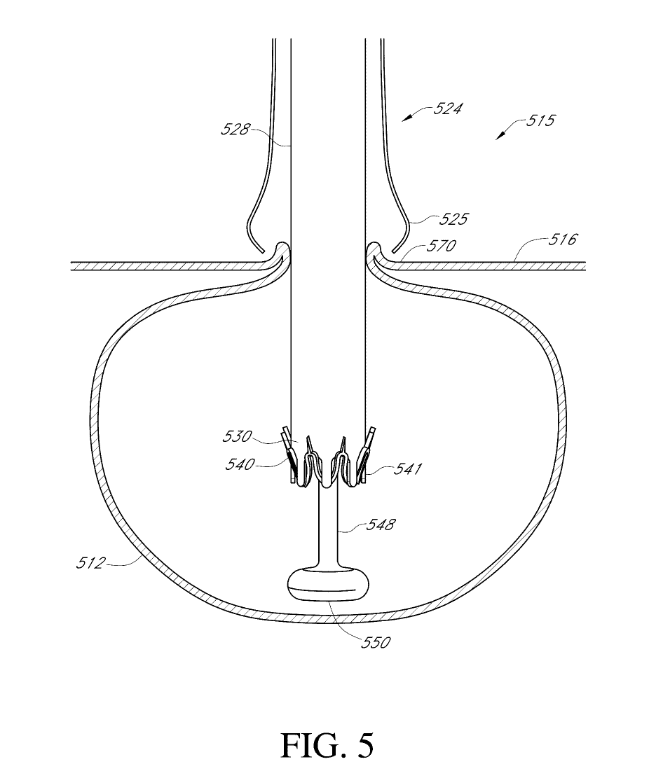

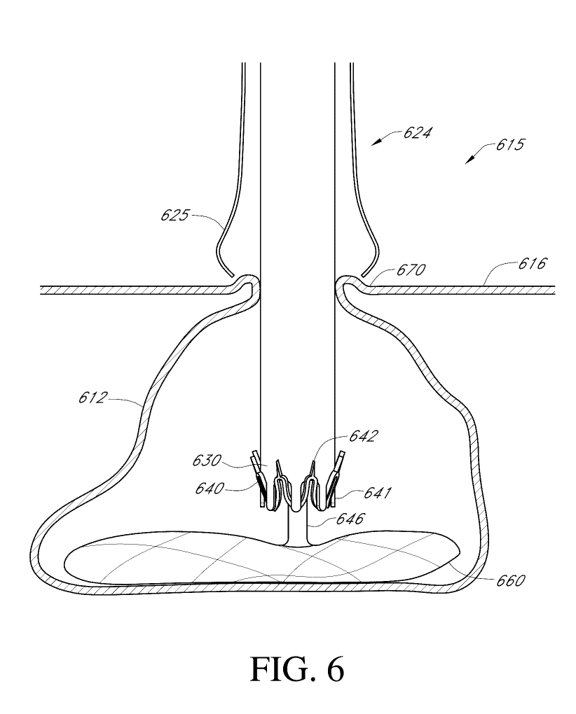

FIGS. 5 and 6 provide embodiments of a device for treating diverticular disease that serves to address the issue discussed above. Both of these embodiments expand the inverted diverticulum prior to the seating and placement of the clip into the target tissue.

In some embodiments, after the ostium of the inverted diverticulum is captured with the distal end of the basket, the distal end of the device can release of burst of gas. The gas can extend the collapsed tissue of the inverted diverticulum. Using gas or fluid can allow the device to accommodate any diverticulum shape or size and independent of the device. Gas input and venting can be provided in separate vials of the concentric tube arrangement of the tool.

FIG. 5 shows a device for diverticulum expansion with fluid release distal end 515. The device for diverticulum expansion with fluid release distal end 515 includes a basket 524 with a plurality of basket tines 525, a clip tube 528, a closure clip 540, and a lumen 548 with a fluid release distal end 550. The basket 524 is disposed about the clip tube 528 and, through the plurality of basket tines 525, gathers the outer tissue wall of colon 516 of the ostium 570 against the outer surface of the clip tube 528. The clip tube 528 is disposed about the lumen 548 and has a clip tube 528 with a plurality of locking structures 530. The closure clip 540 is attached to the distal end of the clip tube 528. As described above in FIG. 5B, the closure clip 540 is configured to attach to the locking structures 530 of the distal end of clip tube 528. Each of the engagement centers 541 of the closure clip 540 engage with the locking structures 530 of the distal end of clip tube 548 such that the clip tines 542 extend outward in a proximal direction. The lumen 548 extends coaxially through the length of the clip tube 528 and can extend beyond both the proximal and distal ends of the clip tube 528. The distal end of the lumen 548 is fluidly connected to the fluid release distal end 550 and the proximal end of the lumen 548 is fluidly connected to a user actuated device (not pictured here).

In operation, the device for diverticulum expansion with fluid release distal end 515 is used after the basket 524 retains the outer tissue wall of colon 516 of the ostium 570 and prior to the seating and placement of the closure clip 540 into the target tissue. Once the fluid release distal end 550 of the lumen 548 is placed in the inverted diverticulum 512, a fluid or gas is introduced into the inverted diverticulum 512 through the lumen 548. In some embodiments the gas introduced can include CO₂, O₂, N₂ and any other biocompatible inert gas. In some embodiments the liquid introduced can be water, saline, or other biocompatible liquid. In some examples the fluid can be a solution that includes anti-inflammatory drugs, growth factors, or antibiotics. The flow of fluid expands the inverted diverticulum 512 and forces the tissue of the inverted diverticulum 512 to approach and contact the outer tissue wall of colon 516. This expansion configures the tissue of the inverted diverticulum 512 and the outer tissue wall of colon 516 to allow a controlled and predictable seating and placing of the closure clip 540 into the target tissue.

In other embodiments, the inverted diverticulum can be expanded either before capture with a mechanical structure. In some examples, this can be in the form of an atraumatic tip at the distal end of a push rod as illustrated above. In other examples, the mechanical structure can be a retractable mechanical expander like a basket that moves tissue of the inverted diverticulum from being collapsed over the clip tines of the closure clip.

Similarly, FIG. 6 shows another embodiment of a device for treating diverticular disease that expands the inverted diverticulum prior to clip placement. FIG. 6 shows a device for diverticulum expansion with expandable distal end 615. The device for diverticulum expansion with expandable distal end 615 includes a basket 624 with a plurality of basket tines 625, a clip tube 628, a closure clip 640, and a push rod 646 with an expandable distal end 660 at the distal end. The basket 624 is disposed about the clip tube 628 and, through the plurality of basket tines 625, gathers the outer tissue wall of colon 616 of the ostium 670 against the outer surface of the clip tube 628. The clip tube 628 is disposed about the push rod 646 and has a distal end of with a plurality of locking structures 630. The closure clip 640 is attached to the distal end of the clip tube 628. The closure clip 640 includes a plurality of engagement centers 641 and clip tines 642. As described above, the closure clip 640 is disposed about the clip tube 628 such that the engagement centers 641 are disposed about the locking structures 630 and the clip tines 642 are flared outwards. The push rod 646 extends coaxially through the length of the clip tube 628 and can extend beyond both the proximal and distal ends of the clip tube 628. The distal end of the push rod 646 is connected to the expandable distal end 660 and the push rod 646 is connected to a user actuated device (not pictured here). Prior to the actuation of the expandable distal end 660, the expandable distal end 660 can be located in a pre-expanded state in the distal end of the clip tube 628. In some embodiments, the expandable distal end 660 is made of an elastic material such as elastic polymer or elastic metal. In some embodiments, the elastic material can be selected from materials such as nitinol, cobalt-chromium, polyurethane, polyethylene terephthalate, and polyethyleneoxide. In some embodiments, the expandable distal end 660 can be in the shape of a braided nitinol cage, a spherical structure, a football structure, a malecot, a spiral sire, or curved wires.

In operation, the device for diverticulum expansion with expandable distal end 615 is used like the embodiment pictured in FIG. 6, except that the expansion of the inverted diverticulum 612 is done by the expandable distal end 660 instead of the release of fluid into the inverted diverticulum 612. In some embodiments, the fluid is a biocompatible liquid such as water or saline. In other examples, the fluid is a solution that can include anti-inflammatory drugs, growth factors, or antibiotics. The expandable distal end 660 expands the inverted diverticulum 612 and, like the release of fluid in the embodiment of FIG. 5, forces the tissue of the inverted diverticulum 612 to approach and contact the outer tissue wall of colon 616. This expansion configures the tissue of the inverted diverticulum 612 and the outer tissue wall of colon 616 to allow a controlled and predictable seating and placement of the closure clip 640 into the target tissue.

While the description generally refers to colonoscopes and treatments within a colon, the devices and methods described herein are not limited to applications within a colon. They can be used to invert and/or treat outpocketings (e.g., diverticula, aneurisms, etc.) in any body lumen. Any reference to a colonoscope should be understood to be applicable to endoscopes generally, and similarly, any reference to a colon should be understood to be applicable to any body lumen.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of clip placement for diverticulum inversion, the method comprising:
positioning the distal end of a clip placement device along an outer wall of a colon at a diverticulum;
inverting the diverticulum into the lumen of the colon with a distal end of a pusher structure within the clip placement device;
advancing a portion of the clip placement device into the inverted diverticulum, wherein the clip placement device comprises the pusher structure, a clip tube, a clip, a clip attachment structure configured to reversibly engage the clip at the distal end of the clip tube, and an expansion apparatus;
expanding the diverticulum with the expansion apparatus such that the volume of the diverticulum increases;
engaging the clip with the diverticulum; and
withdrawing the clip placement device in a proximal direction such that the clip attachment structure disengages the clip from the clip placement device.

2. The method of claim 1 wherein the expansion apparatus is configured to release a gas to expand the diverticulum.

3. The method of claim 2 wherein the gas is an inert and/or biocompatible gas such as CO₂, O₂, or N₂.

4. The method of claim 1 wherein the expansion apparatus is configured to release a fluid to expand the diverticulum.

5. The method of claim 4, wherein the fluid is a biocompatible liquid such as water or saline.

6. The method of claim 4, wherein the fluid is a solution that can include anti-inflammatory drugs, growth factors, or antibiotics.

7. The method of claim 1 wherein the expansion apparatus comprises a retractable structure configured to expand the diverticulum.

8. The method of claim 7 wherein the expansion apparatus comprises an elastic material.

9. The method of claim 8 wherein the elastic material is selected from an elastic polymer or metal such as nitinol, cobalt-chromium, polyurethane, polyethylene terephthalate, and polyethyleneoxide.

10. The method of claim 7 wherein the expansion apparatus can comprise a braided nitinol cage, a spherical structure, a football structure, a malecot, a spiral wire, or curved wires.

11. A clip placement device for diverticulum inversion comprising:
a clip tube;
a clip;
a clip attachment structure, wherein the clip attachment structure is configured to reversibly engage the clip at the distal end of the clip tube; and
an expansion apparatus.

12. The clip placement device of claim 11 wherein the expansion apparatus is configured to release a gas to expand the diverticulum.

13. The method of claim 12 wherein the gas is an inert and/or biocompatible gas such as CO₂, O₂, or N₂.

14. The clip placement device of claim 11 wherein the expansion apparatus is configured to release a fluid to expand the diverticulum.

15. The method of claim 14, wherein the fluid is a biocompatible liquid such as water or saline.

16. The method of claim 14, wherein the fluid is a solution that can include anti-inflammatory drugs, growth factors, or antibiotics.

17. The clip placement device of claim 11 wherein the expansion apparatus comprises a retractable structure configured to expand the diverticulum.

18. The clip placement device of claim 11 wherein the expansion apparatus comprises an elastic material.

19. The clip placement device of claim 18 wherein the elastic material is selected from an elastic polymer or metal such as nitinol, cobalt-chromium, polyurethane, polyethylene terephthalate, and polyethyleneoxide.

20. The clip placement device of claim 11 wherein the expansion apparatus comprises a braided nitinol cage, a spherical structure, a football structure, a malecot, a spiral wire, or curved wires.
